(19) United States
(12) Patent Application Publication
(10) Pub. No.: US 2024/0252781 A1
(43) Pub. Date: Aug. 1, 2024

(54) Infant Mattress Assembly

(71) Applicant: Paul Walker, Newhall, IA (US)

(72) Inventor: Paul Walker, Newhall, IA (US)

(21) Appl. No.: 18/101,920

(22) Filed: Jan. 26, 2023

(51) Int. Cl. A61M 21/02; A47D 15/00; A61H 9/00; A61H 23/02; A61H 23/04

(52) U.S. Cl. CPC A61M 21/02; A47D 15/001; A61H 9/0007; A61H 9/0078; A61H 23/0236; A61H 23/04; A61H 2201/0146; A61H 2201/0207; A61H 2201/0214; A61H 2201/1246; A61H 2201/501; A61H 2201/5048; A61H 2201/5097; A61H 2230/405; A61H 2230/625; A61M 2021/0022; A61M 2021/0061; A61M 2021/0066; A61M 2205/07; A61M 2205/3561; A61M 2230/43

(57) ABSTRACT

An infant mattress assembly includes a box spring that is positionable on a bed frame for supporting the box spring above a floor. A mattress is attached to the box spring and an air chamber is integrated within the mattress. A plurality of vibration units is each attached to the mattress thereby facilitating the plurality of vibration units to vibrate air within the air chamber to soothe the child sleeping on the mattress. A heating element is integrated into the mattress for heating the mattress when the heating element is turned on to warm the child. A control unit is integrated into the box spring and the control unit is in remote communication with a personal electronic device thereby facilitating the personal electronic device to remotely control the vibration and the heating element.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to mattress device and more particularly pertains to a new mattress device for enhancing sleep for a child includes a box spring and mattress which has an air chamber within the mattress. The device includes a plurality of vibrators for vibrating the mattress and a plurality of speakers for emitting soothing sounds for soothing a child to sleep. The device includes a pump for inflating or deflating the air chamber and a heating element for warming the mattress. The device includes motions sensors and a carbon dioxide sensor for sensing the health of the child.

### (2) Description of Related Art Including Information Disclosed Under 37 CFR 1.97 and 1.98

The prior art relates to mattress devices including a baby crib that includes an enclosed and elevated bed and a plurality of sensors for sensing the health of a baby sleeping in the enclosed and elevated bed. The prior art discloses a perforated crib bumper for facilitating a crib to be ventilated thereby reducing risk of sudden infant death syndrome. The prior art discloses a crib with an adjustable frame for lifting or lowering a mattress in the crib. The prior art discloses an infant monitoring device that includes motion sensors for sensing the motion of an infant. The prior art discloses a variety of enclosed bassinette devices that each at least includes biometric sensors for sensing the condition of a sleeping infant.

### BRIEF SUMMARY OF THE INVENTION

An embodiment of the disclosure meets the needs presented above by generally comprising a box spring that is positionable on a bed frame for supporting the box spring above a floor. A mattress is attached to the box spring and an air chamber is integrated within the mattress. A plurality of vibration units is each attached to the mattress thereby facilitating the plurality of vibration units to vibrate air within the air chamber to soothe the child sleeping on the mattress. A heating element is integrated into the mattress for heating the mattress when the heating element is turned on to warm the child. A control unit is integrated into the box spring and the control unit is in remote communication with a personal electronic device thereby facilitating the personal electronic device to remotely control the vibration and the heating element.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of an infant mattress assembly according to an embodiment of the disclosure.

FIG. 2 is a right side view of an embodiment of the disclosure.

FIG. 3 is a cross sectional view taken along line 3-3 of FIG. 2 of an embodiment of the disclosure.

FIG. 4 is a schematic view of an embodiment of the disclosure.

FIG. 5 is a perspective in-use view of an embodiment of the disclosure.

### DETAILED DESCRIPTION OF THE INVENTION

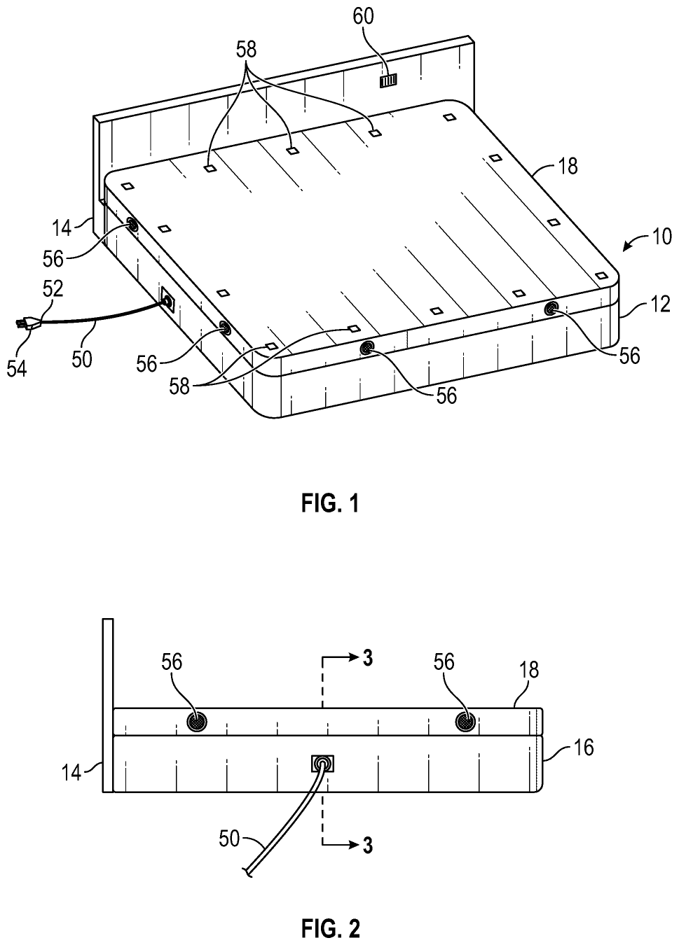

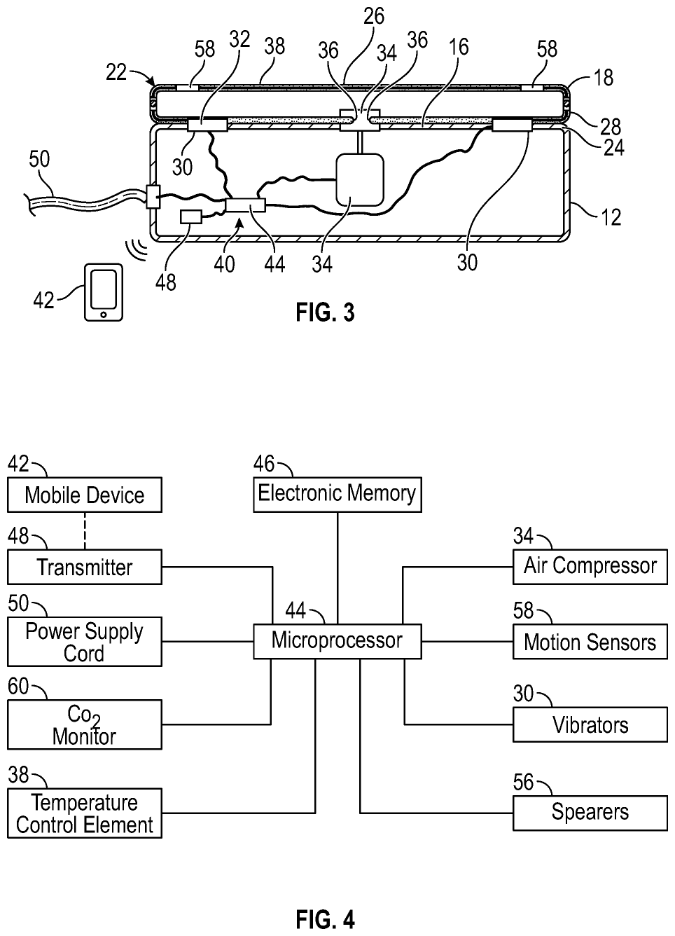

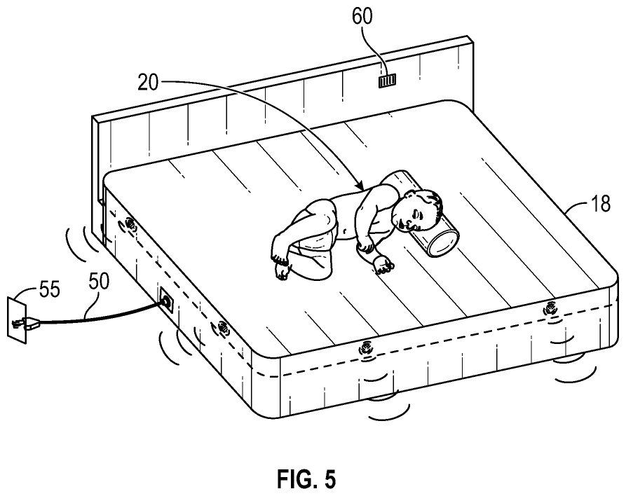

With reference now to the drawings, and in particular to FIGS. 1 through 5 thereof, a new mattress device embodying the principles and concepts of an embodiment of the disclosure and generally designated by the reference numeral 10 will be described.

As best illustrated in FIGS. 1 through 5, the infant mattress assembly 10 generally comprises a box spring 12 that is positionable on a bed frame 14 for supporting the box spring 12 above a floor. The box spring 12 has a top wall 16 and the box spring 12 may be a twin box spring, a queen box spring or any other conventional size of box spring employed for sleeping. A mattress 18 is attached to the box spring 12 thereby facilitating a child 20 to sleep on the mattress 18 and an air chamber 22 is integrated within the mattress 18. The mattress 18 has a lower wall 24, an upper wall 26 and a perimeter wall 28 extending between the lower wall 24 and the upper wall 26. The air chamber 22 is integrated within the lower wall 24 and the upper wall 26 and the perimeter wall 28. Additionally, the lower wall 24 is attached to the top wall 16 of the box spring 12. The mattress 18 may be twin mattress, a queen mattress or any other conventional size of mattress employed for sleeping. The child 20 may be a child that is less than one year old and still at risk of Sudden Infant Death Syndrome.

A plurality of vibration units 30 is each attached between the box spring 12 and the mattress 18 such that each of the plurality of vibration units 30 intrudes into the air chamber 22 in the mattress 18. In this way each of the plurality of vibration units 30 is in communication with an interior of the air chamber 22 thereby facilitating the plurality of vibration units 30 to vibrate air within the air chamber 22 when the plurality of vibration units 30 is turned on. Thus, the plurality of vibration units 30 can soothe the child 20 that is sleeping on the mattress 18. The plurality of vibration units 30 is actuated according to a pre-determined operation sequence thereby facilitating the plurality of vibration units 30 to generate a pulse within the air chamber 22. In this way the plurality of vibration units 30 simulate a mother's heartbeat for the child 20 thereby improving the child's 20 sleep.

Each of the vibration units 30 extends through the top wall 16 of the box spring 12 and the lower wall 24 of the mattress 18. Each of the plurality of vibration units 30 has a vibrating surface 32 that is positioned within the air chamber 22 in the mattress 18. Furthermore, the vibration surface 32 of each of the plurality of vibration units 30 produces a vibration when the plurality of vibration units 30 is turned on. In this way the vibration produced by the vibration surface 32 can be communicated to the air in the air chamber 22 thereby vibrating the mattress 18. The plurality of vibration units 30 is spaced apart from each other and is arranged into a pre-determined pattern for maximizing distribution of the vibration in the mattress 18. Additionally, each of the plurality of vibration units 30 may comprise a subsonic vibrator, a pneumatic vibrator or any other type of vibration source that can impart vibration into air.

A pump 34 is integrated into the box spring 12 and the pump 34 is in fluid communication with the air chamber 22 in the mattress 18. The pump 34 urges air into the air chamber 22 when the pump 34 is actuated into an inflating condition thereby inflating the air chamber 22 for increasing the firmness of the mattress 18. Conversely, the pump 34 urges air outwardly from the air chamber 22 when the pump 34 is actuated into a deflating condition thereby deflating the air chamber 22 for decreasing the firmness of the mattress 18. Additionally, the pump 34 facilitates warmed air within the mattress 18 to be replaced with cooler ambient air thereby cooling the mattress 18 to enhance comfort for the child 20. The pump 34 is attached between the top wall 16 of the box spring 12 and the lower wall 24 of the mattress 18. Additionally, the pump 34 has a pair of output ports 36 that is each in fluid communication with the air chamber 22. The pump 34 may comprise an electric air pump or other similar device.

A heating element 38 is integrated into the mattress 18 and the heating element 38 is in thermal communication with the mattress 18 for heating the mattress 18 when the heating element 38 is turned on to warm the child 20. The heating element 38 is integrated into the upper wall 26 of the mattress 18 and the heating element 38 may comprise an electric heating element 38 with an operational temperature ranging between approximately 60.0 degrees Fahrenheit and 80.0 Fahrenheit. A control unit 40 is integrated into the box spring 12 and the control unit 40 is in communication with each of the plurality of vibration units 30, the pump 34 and the heating element 38. Furthermore, the control unit 40 is in remote communication with a personal electronic device 42 thereby facilitating the personal electronic device 42 to remotely control the vibration units 30 and the pump 34 and the heating element 38.

The control unit 40 comprises a control circuit 44 that is integrated into the box spring 12. The control circuit 44 is electrically coupled to each of the vibration units 30, the pump 34 and the heating element 38. Additionally, the control circuit 44 includes an electronic memory 46 which stores a database comprising a variety of audible sounds. The control unit 40 includes a transceiver 48 that is integrated into the box spring 12. The transceiver 48 is electrically coupled to the control circuit 44 and the transceiver 48 is in wireless communication with the personal electronic device 42. The transceiver 48 may comprise a radio frequency transceiver or the like and the transceiver 48 may employ Bluetooth communication protocols. Furthermore, the personal electronic device 42 may comprise a smart phone or other type of Bluetooth equipped device. The transceiver 48 may be in wireless communication with an existing baby monitor device of any conventional design that is typically employed by a parent to monitor a baby sleeping in an adjacent bedroom.

A power cord 50 is coupled to and extends away from the box spring 12, the power cord 50 is electrically coupled to the control circuit 44 and the power cord 50 has a distal end 52 with respect to the box spring 12. A male plug 54 is electrically coupled to the distal end 52 thereby facilitating the male plug 54 to be plugged into a power source 55 comprising a female electrical outlet. A plurality of speakers 56 is each integrated into the mattress 18 for emitting audible sound outwardly from the mattress 18. Each of the plurality of speakers 56 is electrically coupled to the control circuit 44 thereby facilitating the plurality of speakers 56 to receive the audible sounds from the electronic memory 46. The audible sounds may include the sounds of a womb, a heartbeat or other sounds that a developing infant would hear in their mother's womb. In this way the plurality of speakers 56 can soothe the child 20 to help the child 20 sleep peacefully.

A plurality of motion sensors 58 is provided and each of the plurality of motion sensors 58 is integrated into the upper wall 26 of the mattress 18 to sense motion of the child 20 lying on the mattress 18. Each of the plurality of motion sensors 58 is electrically coupled to the control circuit 44. Additionally, the control circuit 44 is turned on when any of the plurality of motion sensors 58 senses motion. Each of plurality of motion sensors 58 may comprise an electronic motion sensor of any conventional design that has an operational range of less than 1.0 meter.

A carbon dioxide sensor 60 is disposed proximate the mattress 18 to sense carbon dioxide levels in the immediate area surrounding the mattress 18. In this way the carbon dioxide sensor 60 can track the child's 20 respiration. The carbon dioxide sensor 60 is electrically coupled to the control circuit 44 thereby facilitating the transceiver 48 to broadcast the child's 20 respiration to the personal electronic device 42. In this way the carbon dioxide sensor 60 facilitates a caregiver to remotely monitor the child's 20 respiration thereby facilitating the caregiver to immediately respond to a medical emergency involving the child's 20 respiration. The carbon dioxide sensor 60 may comprise an electronic carbon dioxide sensor of any conventional design and the carbon dioxide sensor 60 may be mounted on a headboard that is attached to the box spring 12 or other structural element commonly associated with beds.

In use, the speakers 56, the pump 34 and the vibration units 30 can be remotely controlled with the personal electronic device 42 while the child 20 is sleeping on the mattress 18. In this way the vibration units 30 can vibrate the mattress 18 and the speakers 56 can emit the soothing sounds to soothe the child 20 to sleep. Furthermore, the carbon dioxide sensor 60 monitors the child's 20 respiration and the plurality of motion sensors 58 monitors the child's 20 motion. In this way the parents can monitor the health of the child 20 while the child 20 is sleeping. Furthermore, the speakers 56 and the vibration units 30 improve the quality of sleep for the child 20.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An infant mattress assembly for soothing an infant to sleep as well as monitoring the infant while the infant sleeps, said assembly comprising:
a box spring being positionable on a bed frame for supporting the box spring above a floor;
a mattress being attached to said box spring thereby facilitating a child to sleep on said mattress, said mattress having an air chamber being integrated within said mattress;
a plurality of vibration units, each of said plurality of vibration units being attached between said box spring and said mattress such that each of said plurality of vibration units intrudes into said air chamber in said mattress thereby facilitating each of said plurality of vibration units to be in communication with an interior of said air chamber thereby facilitating said plurality of vibration units to vibrate air within said air chamber when said plurality of vibration units is turned on wherein said plurality of vibration units is configured to soothe the child sleeping on said mattress, said plurality of vibration units being actuated according to a pre-determined operation sequence thereby facilitating said plurality of vibration units to generate a pulse within said air chamber wherein said plurality of vibration units is configured to simulate a mother's heartbeat for the child thereby improving the child's sleep;
a pump being integrated into said box spring, said pump being in fluid communication with said air chamber in said mattress, said pump urging air into said air chamber when said pump is actuated into an inflating condition thereby inflating said air chamber for increasing the firmness of said mattress, said pump urging air outwardly from said air chamber when said pump is actuated into a deflating condition thereby deflating said air chamber for decreasing the firmness of said mattress;
a heating element being integrated into said mattress, said heating element being in thermal communication with said mattress for heating said mattress when said heating element is turned on wherein said heating element is configured to warm the child; and
a control unit being integrated into said box spring, said control unit being in communication with each of said plurality of vibration units and said pump and said heating element, said control unit being in remote communication with a personal electronic device thereby facilitating the personal electronic device to remotely control said vibration units and said pump and said heating element.

2. The assembly according to claim 1, wherein:
said box spring has a top wall;
said mattress has a lower wall, an upper wall and a perimeter wall extending between said lower wall and said upper wall, said air chamber being integrated within said lower wall and said upper wall and said perimeter wall, said lower wall being attached to said top wall of said box spring;
each of said vibration units extends through said top wall of said box spring and said lower wall of said mattress;
said pump is attached between said top wall of said box spring and said lower wall of said mattress;
said heating element is integrated into said upper wall of said mattress; and
said pump has a pair of output ports each being in fluid communication with said air chamber.

3. The assembly according to claim 2, wherein:
each of said plurality of vibration units has a vibrating surface being positioned within said air chamber in said mattress, said vibration surface of each of said plurality of vibration units producing a vibration when said plurality of vibration units is turned on thereby facilitating said vibration produced by said vibration surface to be communicated to the air in said air chamber thereby vibrating said mattress; and
said plurality of vibration units is spaced apart from each other and arranged into a pre-determined pattern for maximizing distribution of said vibration in said mattress.

4. The assembly according to claim 1, wherein said control unit comprises:
a control circuit being integrated into said box spring, said control circuit being electrically coupled to each of said vibration units and said pump and said heating element, said control circuit including an electronic memory storing a database comprising a variety of audible sounds;
a transceiver being integrated into said box spring, said transceiver being electrically coupled to said control circuit, said transceiver being in wireless communication with the personal electronic device; and
a power cord being coupled to and extending away from said box spring, said power cord being electrically coupled to said control circuit, said power cord having a distal end with respect to said box spring, said power cord having a male plug being electrically coupled to said distal end thereby facilitating said male plug to be plugged into a power source comprising a female electrical outlet.

5. The assembly according to claim 4, further comprising a plurality of speakers, each of said speakers being integrated into said mattress wherein said plurality of speakers is configured to emit audible sound outwardly from said mattress, each of said plurality of speakers being electrically coupled to said control circuit thereby facilitating said plurality of speakers to receive the audible sounds from said electronic memory comprising sounds of a womb wherein said plurality of speakers is configured to soothe the child.

6. The assembly according to claim 4, further comprising a plurality of motion sensors, each of said plurality of motion sensors being integrated into an upper wall of said mattress wherein each of said plurality of motion sensors is configured to sense motion of the child lying on said mattress, each of said plurality of motion sensors being electrically coupled to said control circuit, said control circuit being turned on when any of said plurality of motion sensors senses motion.

7. The assembly according to claim 4, further comprising a carbon dioxide sensor being disposed proximate said mattress wherein said carbon dioxide sensor is configured to sense carbon dioxide levels in the immediate area surrounding said mattress thereby facilitating said carbon dioxide sensor to track the child's respiration, said carbon dioxide sensor being electrically coupled to said control circuit thereby facilitating said transceiver to broadcast the child's respiration to the personal electronic device wherein said carbon dioxide sensor is configured to facilitate a caregiver to remotely monitor the child's respiration thereby facilitating the caregiver to immediately respond to a medical emergency involving the child's respiration.

8. An infant mattress assembly for soothing an infant to sleep as well as monitoring the infant while the infant sleeps, said assembly comprising:
a box spring being positionable on a bed frame for supporting the box spring above a floor, said box spring having a top wall;
a mattress being attached to said box spring thereby facilitating a child to sleep on said mattress, said mattress having an air chamber being integrated within said mattress, said mattress having a lower wall, an upper wall and a perimeter wall extending between said lower wall and said upper wall, said air chamber being integrated within said lower wall and said upper wall and said perimeter wall, said lower wall being attached to said top wall of said box spring;
a plurality of vibration units, each of said plurality of vibration units being attached between said box spring and said mattress such that each of said plurality of vibration units intrudes into said air chamber in said mattress thereby facilitating each of said plurality of vibration units to be in communication with an interior of said air chamber thereby facilitating said plurality of vibration units to vibrate air within said air chamber when said plurality of vibration units is turned on wherein said plurality of vibration units is configured to soothe the child sleeping on said mattress, said plurality of vibration units being actuated according to a pre-determined operation sequence thereby facilitating said plurality of vibration units to generate a pulse within said air chamber wherein said plurality of vibration units is configured to simulate a mother's heartbeat for the child thereby improving the child's sleep, each of said vibration units extending through said top wall of said box spring and said lower wall of said mattress, each of said plurality of vibration units having a vibrating surface being positioned within said air chamber in said mattress, said vibration surface of each of said plurality of vibration units producing a vibration when said plurality of vibration units is turned on thereby facilitating said vibration produced by said vibration surface to be communicated to the air in said air chamber thereby vibrating said mattress, said plurality of vibration units being spaced apart from each other and being arranged into a pre-determined pattern for maximizing distribution of said vibration in said mattress;
a pump being integrated into said box spring, said pump being in fluid communication with said air chamber in said mattress, said pump urging air into said air chamber when said pump is actuated into an inflating condition thereby inflating said air chamber for increasing the firmness of said mattress, said pump urging air outwardly from said air chamber when said pump is actuated into a deflating condition thereby deflating said air chamber for decreasing the firmness of said mattress, said pump being attached between said top wall of said box spring and said lower wall of said mattress, said pump having a pair of output ports each being in fluid communication with said air chamber;
a heating element being integrated into said mattress, said heating element being in thermal communication with said mattress for heating said mattress when said heating element is turned on wherein said heating element is configured to warm the child, said heating element being integrated into said upper wall of said mattress;
a control unit being integrated into said box spring, said control unit being in communication with each of said plurality of vibration units and said pump and said heating element, said control unit being in remote communication with a personal electronic device thereby facilitating the personal electronic device to remotely control said vibration units and said pump and said heating element, said control unit comprising:
a control circuit being integrated into said box spring, said control circuit being electrically coupled to each of said vibration units and said pump and said heating element, said control circuit including an electronic memory storing a database comprising a variety of audible sounds;
a transceiver being integrated into said box spring, said transceiver being electrically coupled to said control circuit, said transceiver being in wireless communication with the personal electronic device; and
a power cord being coupled to and extending away from said box spring, said power cord being electrically coupled to said control circuit, said power cord having a distal end with respect to said box spring, said power cord having a male plug being electrically coupled to said distal end thereby facilitating said male plug to be plugged into a power source comprising a female electrical outlet;
a plurality of speakers, each of said speakers being integrated into said mattress wherein said plurality of speakers is configured to emit audible sound outwardly from said mattress, each of said plurality of speakers being electrically coupled to said control circuit thereby facilitating said plurality of speakers to receive the audible sounds from said electronic memory comprising sounds of a womb wherein said plurality of speakers is configured to soothe the child;
a plurality of motion sensors, each of said plurality of motion sensors being integrated into said upper wall of said mattress wherein each of said plurality of motion sensors is configured to sense motion of the child lying on said mattress, each of said plurality of motion sensors being electrically coupled to said control circuit, said control circuit being turned on when any of said plurality of motion sensors senses motion; and
a carbon dioxide sensor being disposed proximate said mattress wherein said carbon dioxide sensor is configured to sense carbon dioxide levels in the immediate area surrounding said mattress thereby facilitating said carbon dioxide sensor to track the child's respiration, said carbon dioxide sensor being electrically coupled to said control circuit thereby facilitating said transceiver to broadcast the child's respiration to the personal electronic device wherein said carbon dioxide sensor is configured to facilitate a caregiver to remotely monitor the child's respiration thereby facilitating the caregiver to immediately respond to a medical emergency involving the child's respiration.
